(19) United States
(12) Patent Application Publication
(10) Pub. No.: US 2005/0196354 A1
(43) Pub. Date: Sep. 8, 2005

(54) Film compositions

(76) Inventor: Andre Soshinsky, Randolph, NJ (US)

Correspondence Address: Warner-Lambert Company LLC, 201 Tabor Road, Morris Plains, NJ, 07950, US

(21) Appl. No.: 10/792,066

(22) Filed: Mar. 3, 2004

(51) Int. Cl. A61K 7/16; A61K 7/20

(52) U.S. Cl. 424/53; 424/443

(57) ABSTRACT

This invention relates generally to film compositions for use in the delivering topical and/or systemic actives, and more particularly to a slow dissolving or disintegrating strips, especially for delivering oral agents to the teeth and gums.

### FIELD OF THE INVENTION

This invention relates generally to film compositions for use in the delivering topical and/or systemic actives, and more particularly to a slow dissolving or disintegrating strips, especially for delivering oral agents to the teeth and gums.

### BACKGROUND OF THE INVENTION

Delivery devices are well known for delivering oral care actives to the surface of the teeth and/or the oral tissue of the mouth. These devices typically take the form of a flexible, oral strip or film comprising an adhesive surface and a suitable oral care active.

The most notable of such film compositions relates to the over-the-counter teeth whitening systems now available, including a whitening system comprised of a thin strip of plastic film having applied to a surface thereof a tooth whitening composition as described in U.S. Pat. Nos. 5,894,017, 5,891,453 and 6,045,811.

A problem with such film compositions, however, relates to the dissolution rate of the film compositions and, more specifically, to controlling the dissolution rate of such film compositions. Ideally, the film composition should not be present in the mouth for long periods, but dissolve or disintegrate slow enough such that the oral care active has time to exert its activity. Moreover, controlling film dissolution rates also facilitates the improvement of other film benefits such as barrier protection.

The present inventor has found that by manipulating the individual water-soluble layers forming a multi-layered film, improved control over the dissolution rate of the multi-layered film is achieved. Specifically, the present inventor has discovered that the dissolution rate of multilayered films can be controlled by superimposing at least two (2) water soluble layers. The components of these superimposed layers interact with each other to form new component with much lower dissolution or disintegration rate. The at least two (2) layers can be formed any number of ways, for example, by simply using oppositely charged water soluble polymers to produce the respective layers. Or, alternatively, the at least two layers can be formed by adding at least one polyvalent cationic ion source to at least one neutrally charged water soluble polymer to form one layer and using a negatively charged water soluble polymer to form another layer. The interacting layers can also be formed by superimposing a water soluble layer comprising either an anionic or cationic surfactant and a neutrally charged polymer with a water soluble layer comprising a neutrally charged polymer and a water soluble salt.

Accordingly an aspect of the present invention is to provide film products which dissolve slowly in the oral cavity.

Another aspect of the present invention is to provide film products comprising interacting water-soluble layers which when combined result in multi-layered film products having a slower dissolution rate than any of the individual film layers used in forming it.

Still one other aspect of the present invention is to provide slow dissolving film products for delivering topical or systemic actives.

Still yet one other aspect of the present invention is to provide film products for delivering topical or systemic actives wherein the film dissolves or disintegrates within 1 minute to 12 hours, optionally within 3 minutes to 6 hours or, optionally, within 10 minutes to 1 hour in an aqueous environment.

One more aspect of the invention is the use of water soluble materials in both layers so as not to require organic solvents, which reduces costs, and improves safety of manufacturing.

### SUMMARY OF THE INVENTION

In one embodiment, the present invention relates to multi-layer, stand alone, film compositions, comprising:
a.) a first water-soluble layer comprising:
   i.) a neutral, water soluble polymer; and
   ii.) a polyvalent cationic ion source; and
b.) a second water-soluble layer comprising anionic water soluble polymer;
   wherein the multi-layer film composition dissolves or disperses in water at a rate slower than any of the individual water-soluble layers.

In another embodiment, the present invention relates to multi-layer, stand alone, film compositions, comprising:
a.) a first water-soluble layer comprising a cationic polymer; and
b.) a second water-soluble layer comprising a anionic polymer
   wherein the multi-layer film composition dissolves or disperses in water at a rate slower than any of the individual water-soluble layers.

In still another embodiment, the present invention relates to multi-layer, stand alone, film compositions, comprising:
a.) a first water-soluble layer comprising:
   a.) a neutral water soluble polymer; and
   ii.) an anionic surfactant; and
b.) a second water-soluble layer comprising a cationic water soluble polymer
   wherein the multi-layer film composition dissolves or disperses in water at a rate slower than any of the individual water-soluble layers.

In still another embodiment, the present invention relates to multi-layer, stand alone, film compositions, comprising:
a.) a first water-soluble layer comprising:
   i.) a neutral water soluble polymer; and
   ii.) a cationic surfactant; and
b.) a second water-soluble layer comprising an anionic water soluble polymer;
   wherein the multi-layer film composition dissolves or disperses in water at a rate slower than any of the individual water-soluble layers.

In yet one other embodiment, the present invention relates to multi-layer, stand alone, film compositions, comprising:
a.) a first water-soluble layer comprising:
   i.) a neutral water soluble polymer; and
   ii.) an anionic or a cationic surfactant; and
b.) a second water-soluble layer comprising:
   i.) a neutral water soluble polymer;
   ii.) water-soluble salt.
      wherein the multi-layer film composition dissolves or disperses in water at a rate slower than any of the individual water-soluble layers.

Methods of manufacturing and using are also disclosed.

### DETAILED DESCRIPTION OF THE PRESENT INVENTION

The film compositions of the present invention can comprise, consist of, or consist essentially of the essential elements and limitations of the invention described herein, as well any of the additional or optional ingredients, components, or limitations described herein.

All percentages, parts and ratios are based upon the total weight of the wet film composition of the present invention, unless otherwise specified. All such weights as they pertain to the listed ingredients are based on the active level and, therefore, do not include carriers or by-products that may be included in commercially available materials, unless otherwise specified.

The term “safe and effective amount” as used herein means an amount of a compound or composition such as a topical or system active sufficient to significantly induce a positive benefit, for example, a teeth whitening, antimicrobial and/or analgesic benefit, including independently the benefits disclosed herein, but low enough to avoid serious side effects, i.e., to provide a reasonable benefit to risk ratio, within the scope of sound judgment of the skilled artisan.

The term “adhesive” as used herein, means any material or composition that is capable of sticking to the site of topical application or administration and includes, but is no limited to, mucoadhesives, pressure-sensitive adhesive (adheres upon application of pressure), moistenable adhesives (adheres in the presence of water) and tacky or sticky type adhesives (adheres upon immediate contact with a surface).

The phrase “charged entity” as used herein includes, but is not limited to, anionic or cationic polymers, anionic or cationic surfactants, water soluble salts, polyvalent cationic sources and mixtures thereof.

The term “foreign substances” as used herein means dirt, infectious microorganisms and the like.

Optionally, the film compositions of the present invention are clear. The term “clear” as defined herein ranges from transparent to translucent as observed with the naked eye.

The film compositions of the present invention, including the essential and optional components thereof, are described in detail hereinafter.

The Water Soluble Polymer

Depending on the particular embodiments in mind, non-ionic or charge neutral polymers, cationic or anionic water soluble polymers can be used in forming the film compositions of the present invention.

Examples of suitable water soluble polymers include, but are not limited to, alkylcelluloses such as methylcellulose, hydroxyalkylcelluloses such as hydroxybutyl cellulose, hydroxylethylmethyl cellulose, hydroxyethylcellulose and hydroxypropylcellulose; hydroxyalkyl alkylcelluloses such as hydroxypropyl methylcellulose; carboxyalkylcelluloses such as carboxymethylcellulose; alkali metal salts of carboxyalkylcelluloses such as sodium carboxymethylcellulose; carboxyalkylalkylcelluloses such as carboxymethylethylcellulose; carboxyalkylcellulose esters; starches; pectins such as sodium carboxymethylamylopectin; chitin derivatives such as chitosan; cationic polymers such as polyquaternium-10, polyquaternium-16, polyquaternium-28, polyquaternium-44, polyquaternium-46, polyquaternium-55, vinylpyrrolidone/dimethylaminopropyl methacrylamide copolymer, vinylpyrrolidone/dimethylaminoethyl methacrylate copolymer; polysaccharides such as alginic acid, alkali metal and ammonium salts thereof, carrageenans, galactomannans, traganth, agar, gum arabicum, guar gum and xanthan gum; polyacrylic acids and salts thereof; polymethacrylic acids and salts thereof, including methacrylate-vinyl alcohol copolymers, polyvinyl alcohol, polyvinyl alcohol copolymers or derivatives, vinyl acetate-vinyl alcohol copolymers, polyvinylpyrrolidone, hydrolyzed polyvinylpyrrolidone, polyacrylamide, poly(methacrylamide), dextran, polyethylene glycol, and polyoxyethylene and polyoxypropylene block copolymers and mixtures thereof.

Non-Ionic Polymers

The nonionic water-soluble polymers are, for example, hydroxyethyl cellulose, hydroxypropyl cellulose, hydroxypropyl methylcellulose, methylcellulose, polyvinyl alcohol, polyvinyl alcohol copolymers or derivatives, vinyl acetate-vinyl alcohol copolymers, polyvinylpyrrolidone, hydrolyzed polyvinylpyrrolidone, polyacrylamide, poly(methacrylamide), dextran, polyethylene glycol, and polyoxyethylene and polyoxypropylene block copolymers and mixtures thereof.

In certain embodiments, the neutral or non-ionic water soluble polymers include, but are not limited to hydroxyethyl cellulose (HEC), hydroxypropyl cellulose (HPC), polyvinylpyrrolidone (PVP), polyvinylpyrrolidone/vinyl acetate copolymer (PVP/VA), hydroxypropyl methylcellulose (HPMC), methylcellulose (MC), polyethylene glycol (PEG), polyvinyl alcohol (PVA), and the mixtures thereof.

Suitable HEC, HPC, HPMC and MC polymers are supplied by Hercules, Wilmington, Del. Suitable PVP and PVP/VA polymers are supplied by International Specialties Products (ISP), Wayne, N.J. Suitable PVA polymers are supplied by DuPont, Wilmington, Del. And, suitable PEG polymers are supplied by BASF, Mount Olive, N.J.

When incorporated in the film compositions of the present invention, the neutral or non-ionic water soluble polymer is present at a concentration of from about 0.1% to about 50% optionally, from about 0.2% to about 40%, and, optionally, from about 0.5% to about 30%, by weight, of the wet film composition.

Anionic Polymers

Anionic water-soluble polymers according to the invention include, but are not limited to, sodium carboxymethylcellulose, sodium carboxymethyl hydroxyethylcellulose, pectin, carrageenan, carboxymethylguar gum, sodium alginate, anionic polyacrylamide copolymers, alkali-soluble latex, carboxymethyl methylcellulose, carboxymethyl hydroxypropyl guar, and other anionic carbohydrate derivatives, as well as mixtures including one or more of these polymers. Other suitable anionic polymers include polymaleic acid, polysulfonates, and mixtures thereof. Additional anionic polymeric polycarboxylates, such as Gantrez, are described in U.S. Pat. No. 5,037,637 to Gaffar et al, issued Aug. 6, 1991, herein incorporated by reference in its entirety including all references incorporated into this reference. In certain embodiments, the anionic polymer is a carboxyvinyl polymer. Carboxyvinyl polymers are described in U.S. Pat. No. 2,798,053 to Brown, issued Jul. 2, 1957, herein incorporated by reference in its entirety. Carboxyvinyl polymers are provided by B. F. Goodrich Company as Carbopol® 934, 940, 941, 946, and 956. Mixtures of these anionic polymers can also be used herein.

Commercially available products which may be used as the anionic water-soluble first ionic polymer, or as a component thereof, include CMC-9M31 (sodium carboxymethylcellulose; Hercules Incorporated), CMHC 420H (carboxymethyl hydroxyethylcellulose; Hercules Incorporated), Pectin LM104 AS-Z (anionic pectin; Hercules Incorporated), Gelcarin GP 911 brand carrageenan (FMC Biopolymer, Philadelphia, Pa.), Galactosol (carboxymethyl guar gum, Hercules Incorporated), Alcogum L-29 (an alkali-soluble latex; Alco Products), Kelgin MV (sodium alginate; Kelco, San Diego), Reten 215 (anionic polyacrylamide; Hercules Incorporated) or mixtures thereof.

In certain embodiments, the anionic polymer includes sodium carboxymethylcellulose (supplied by Hercules, Wilmington, Del.), pectin (supplied by Hercules, Wilmington, Del.), carrageenan, sodium alginate (supplied Keltone HVCR, ISP Alginates, San Diego, Calif.), sodium polyacrylate (supplied as Nihon Junyaku, Tokio, Japan), sodium polymethacrylate (supplied as Darvan #7, R. T. Vanderbilt, Norwalk, Conn.), sodium maleate/methyl vinyl ether copolymer (supplied as Gantrez S-97, ISP, Wayne, N.J.) as well as mixtures including one or more of these polymers.

When incorporated in the film compositions of the present invention, the anionic water soluble polymer is present at a concentration of from about 0.01% to about 50% optionally, from about 0.1% to about 40%, and, optionally, from about 0.4% to about 30%, by weight, of the wet film composition.

Cationic Polymers

Suitable water soluble cationic polymers are, for example, cationic cellulose derivatives such as, for example, the quaternized hydroxyethyl cellulose obtainable from Amerchol under the name of Polymer JR 400®; cationic starch; copolymers of diallyl ammonium salts and acrylamides; quaternized vinyl pyrrolidone/vinyl imidazole polymers such as, for example, Luviquat®, (BASF); condensation products of polyglycols and amines; quaternized collagen polypeptides such as, for example, lauryldimonium hydroxypropyl hydrolyzed collagen (Lamequat® L, Grunau GmbH); quaternized wheat polypeptides; polyethyleneimine; cationic silicone polymers such as, for example, Amidomethicone; copolymers of adipic acid and dimethylaminohydroxypropyl diethylenetriamine (Cartaretine® Sandoz AG); polyacrylamide polymers and copolymers; copolymers of acrylic acid with dimethyl diallyl ammonium chloride (Merquat® 550, Chemviron); polyaminopolyamides as described, for example, in FR-A 2 252 840; epichlorohydrin\dimethylamine polymers (EPI-DMA) or epihalohydrin reaction products of polyaminoamides obtained by reaction of polyamines with dicarboxylic acids and crosslinked water-soluble polymers thereof; diallyldimethyl ammonium chloride (DADMAC) and polymers of DADMAC such as DADMAC/acrylamide copolymers; cationic chitin derivatives such as, for example, quaternized chitosan, optionally in microcrystalline distribution, condensation products of dihaloalkyls, for example dibromo butane, with bis-dialkylamines, for example bis-dimethylamino-1,3-propane, cationic guar gum such as, for example, Jaguar® CBS, Jaguar®C-17, Jaguar®(C-16 of Celanese, USA, quaternized ammonium salt polymers such as, for example, Mirapol®A-15, Mirapol®AD-1, Mirapol®AZ-1 of Miranol, USA; ionene polymers and mixtures thereof.

Examples of ionene polymers useful herein include, but are not limited to, those set forth in U.S. Pat. Nos. 5,681,862 and 5,575,993, both of which are incorporated by reference in their entirety. Further, the polymers set forth in U.S. Pat. No. 5,256,252 can also be used herein and is incorporated herein by reference in its entirety.

In certain embodiments, the cationic water soluble polymer includes, but is not limited to, chitosan (supplied as Protasan UP CL213, FMC Biopolymer, Philadelphia, Pa.), polyquatemium-10, polyquatemium-11 (ISP, Wayne, N.J.)., polyquatemium-16 (BASF, Mount Olive, N.J.), polyquaternium-24 (Amerchol, Edison, N.J.), polyquaternium-28 (ISP, Wayne, N.J.)., polyquatemium-44 (BASF, Mount Olive, N.J.), polyquatemium-46 (BASF, Mount Olive, N.J.), polyvinylpyrrolidone/dimethylaminopropyl methacrylamide copolymer (ISP, Wayne, N.J.)., and the mixtures thereof.

When incorporated in the film compositions of the present invention, the cationic water soluble polymer is present at a concentration of from about 0.01% to about 50% optionally, from about 0.1% to about 40%, and, optionally, from about 0.4% to about 30%, by weight, of the wet film composition.

When incorporated into certain embodiments of the present invention, the ratio of the anionic water-soluble polymer to the cationic water-soluble polymer is from about 20:1 to about 1:20, optionally, from about 5:1 to about 1:5 and, optionally, from about 2:1 to about 1:2.

The Polyvalent Cationic Ion Source

Incorporated in certain embodiments of the compositions of the present invention is a polyvalent cationic ion source or salt for providing cationic ions of at least two positive charges such as alkaline-earth metals, transition metals, or mixtures thereof.

Useful polyvalent cationic ion sources include, but are not limited to, water soluble salts such as aluminum salts, chromium salts, calcium salts, zinc salts, magnesium salts, iron salts, barium salts, manganese salts, stannous salts and the mixtures thereof. Specific water soluble salts include, but are not limited to, stannous chloride, manganese chloride, calcium chloride, magnesium chloride, calcium nitrate, magnesium nitrate, and mixtures thereof.

When incorporated in the film compositions of the present invention, the cationic ion source is present at a concentration of from about 0.001% to about 10% optionally, from about 0.01% to about 5%, and, optionally, from about 0.02% to about 2%, by weight, of the wet film composition.

Water Soluble Salts

Incorporated in certain embodiments of the compositions of the present invention are water soluble salts such as sodium chloride, potassium chloride, magnesium chloride, calcium chloride, zinc chloride, sodium acetate, sodium fluoride, sodium phosphate, potassium phosphate, sodium citrate, sodium oxalate, acidic salts thereof, and mixtures thereof.

When incorporated in the film compositions of the present invention, the water soluble salt is present at a concentration of from about 0.001% to about 10% optionally, from about 0.01% to about 5%, and, optionally, from about 0.02% to about 2%, by weight, of the wet film composition.

Anionic Surfactants

Anionic surfactants useful herein include, but are not limited to, the water-soluble salts of alkyl sulfates having from 8 to 20 carbon atoms in the alkyl radical (e.g., sodium alkyl sulfate) and the water-soluble salts of sulfonated monoglycerides of fatty acids having from 8 to 20 carbon atoms. Sodium lauryl sulfate (Rhodia Inc, Cranbury, N.J.) and sodium coconut monoglyceride sulfonates are examples of anionic surfactants of this type. Other suitable anionic surfactants are sarcosinates, such as sodium lauryl sarcosinate, taurates such as sodium cocoyl taurates, sodium lauryl sulfoacetate, sodium lauroyl isethionate, sodium laureth carboxylate, and sodium dodecyl benzenesulfonate (Witco, Houston, Tex.). Mixtures of anionic surfactants can also be employed. Many suitable anionic surfactants are disclosed in U.S. Pat. No. 3,959,458, to Agricola, et al., incorporated by reference herein in its entirety by reference.

In certain embodiments, the anionic surfactants include, but are not limited to, sodium lauryl sulfate, sodium dodecyl benzenesulfonate, sodium C12-15 pareth-15 sulfonate (BASF, Mount Olive, N.J.)., sodium methyl cocoyl taurate (Croda, Parsippany, N.J.), disodium lauryl sulfosuccinate (McIntire Group, University Park, Ill.), and the mixtures thereof.

When incorporated in the film compositions of the present invention, the anionic surfactant is present at a concentration of from about 0.001% to about 10%, optionally, from about 0.005% to about 5%, and, optionally, from about 0.01% to about 2%, by weight, of the wet film composition.

Cationic Surfactants

Cationic surfactants useful as cationic sources include, but not limited quaternary ammonium compounds such as cetyl pyridinium chloride, benzalkonium chloride, benzethonium chloride, cetrimonium bromide, stearyl dimethylbenzyl ammonium chloride, dodecyl trimethylammonium chloride, polyoxyethylene and coconut amine and mixtures thereof.

In certain embodiments, the cationic surfactant includes, but is not limited to, cetyl pyridinium chloride (Spectrum Laboratory Products ,Gardena, Calif.), benzalkonium chloride (Lonza, Fairlawn, N.J.), dodecyl trimethylammonium chloride (Mallinckrodt Baker, Philipsburg, N.J.), and the mixtures thereof.

When incorporated in the film compositions of the present invention, the cationic surfactant is present at a concentration of from about 0.001% to about 10%, optionally, from about 0.005% to about 5%, and, optionally, from about 0.01% to about 2%, by weight, of the wet film composition.

Method of Preparing the Film Compositions

The multilayer films can be prepared accordingly. In one embodiment, a polymer solution containing a neutral or nonionic polymer, an anionic or a cationic surfactant and desired active ingredients (where appropriate, together with auxiliaries) is produced. This solution is coated onto an inert processing sheet made of plastic or metal by knife or roller application or spraying processes. Drying subsequently results in an initial film, also called the basic layer. In the next step, a second polymer solution containing a neutral or nonionic polymer, a water soluble salt and desired active ingredients (where appropriate, together with auxiliaries) is produced. The second polymer solution is cast over the basic layer and allowed to dry, resulting in a tri-layer composite. Without being limited by theory, it is believed that an interface or intermediate complex layer is formed between the combined water soluble layers such that the complex layer is less soluble than the individual layers which are combined to form the interface or intermediate complex layer, resulting in the overall decreased solubility and corresponding increased retention time of the multilayer film. It is further believed, without being limited by theory, that the interface or intermediate complex layer results from the interaction between the charged entities provided by each layer and, optionally, the hydrophobic interactions provided by the surfactant and/or polymer molecules.

By manipulating the concentrations and ratios of water soluble neutral polymers, salt(s), surfactant(s), the dissolution rates of the bi- or multi-layer films can be adjusted faster or slower. Thicknesses of the layers being combined are additional parameters that can be used to manipulate the dissolution rate of resulting film.

Optionally, one or more subsequent polymer solution layers can be applied to one or the other of this tri-layer composite. The third layer added in this case can have either the same composition as the second, so that a symmetrical structure results, or a different one.

Alternatively, a polymer solution containing an anionic or cationic polymer and desired active ingredients (where appropriate, together with auxiliaries) is produced. This solution is coated onto an inert processing sheet made of plastic or metal by knife or roller application or spraying processes. Drying subsequently results in an initial film, also called the basic layer. In the next step, a second polymer solution containing a polymer having a charge opposite the polymer in the basic layer (or a neutrally charged polymer in admixture with a cationic or anionic surfactant or a polyvalent ion source having a charge opposite the charge of the polymer in the basic layer) and desired active ingredients (where appropriate, together with auxiliaries) is produced. The second polymer solution is cast over the basic layer and allowed to dry, resulting in a tri-layer composite. In this same way, water soluble layers containing cationic water soluble polymers can, likewise, be added to water soluble layers formed from a neutrally charged water soluble polymer in admixture with an anionic surfactant. In this same way, water soluble layers containing anionic water soluble polymers can, likewise, be added to water soluble layers formed from a neutrally charged water soluble polymer in admixture with a cationic surfactant or a polyvalent cationic ion source.

The drying step can be accomplished at room temperature or by heating.

By manipulating the concentrations and ratios of water soluble neutral, cationic or anionic polymers, salt(s), and surfactant(s), the dissolution rates of the bi- or multi-layer films can be adjusted faster or slower. Thicknesses of the layers being combined are additional parameters that can be used to manipulate the dissolution rate of resulting film.

Additional layers can added as taught above.

The processes according to the invention can be carried out both continuously and batchwise. The inert substrates used for continuous processes are preferably processing sheets or metal sheets or strips, whereas in batchwise processes it is possible in principle to use any inert substrates.

Optional Ingredients

Various topical and systemic actives can also be incorporated into the films of the present invention. The term “topical or system active” as used herein includes curative, prophylactic and cosmetic active substances or compositions thereof. Examples of the conditions these substances may address include, but are not limited to one or more of, appearance and structural changes to teeth, whitening, stain bleaching, stain removal, plaque removal, tartar removal, cavity prevention and treatment, inflamed and/or bleeding gums, mucosal wounds, lesions, ulcers, aphthous ulcers, cold sores, tooth abscesses, tooth and/or gum pain, tooth sensitivity (e.g. to temperature changes), and the elimination of mouth malodour resulting from the conditions above and other causes such as microbial proliferation. Additionally, the films of the present invention are useful for treating and/or preventing wounds, lesions, ulcers, cold sores and the like of the lips and skin generally.

Suitable topical actives for use in and around the oral cavity include any substance that is generally considered as safe for use in the oral cavity and that provides a change to the overall health of the oral cavity. The level of topical oral care active in the present invention may generally be from about 0.01% to about 40% or, optionally, from about 0.1% to 20% by weight of the wet film.

The topical oral care actives of the present invention may include many of the actives previously disclosed in the art. The following is a non all-inclusive list of oral care actives that may be used in the present invention.

Essential oils may be included in or associated with the films the present invention. Essential oils suitable for use herein are described in detail in U.S. Pat. Nos. 6,596,298 to Leung et al., previously incorporated by reference in its entirety.

Teeth whitening actives may be included in the films of the present invention. The actives suitable for whitening are selected from the group consisting of oxalates, peroxides, metal chlorites, perborates, percarbonates, peroxyacids, and mixtures thereof. Suitable peroxide compounds include: hydrogen peroxide, calcium peroxide, sodium peroxide, carbamide peroxide, urea peroxide, sodium percarbonate and mixtures thereof. Optionally, the peroxide is hydrogen peroxide. Suitable metal chlorites include calcium chlorite, barium chlorite, magnesium chlorite, lithium chlorite, sodium chlorite and potassium chlorite. Additional whitening actives may be hypochlorite and chlorine dioxide. A preferred chlorite is sodium chlorite. The effectiveness of whitening actives can, optionally, be enhanced by means of a catalyst, i.e. a two-component peroxide-catalyst; system. Useful whitening agent catalysts or catalytic agents can be found in U.S. Pat. No. 6,440,396 to McLaughlin, Gerald, herein incorporated by reference in its entirety.

When incorporating peroxide actives, the film compositions of the present invention can, optionally, contain peroxide active stabilizers. Peroxide active stabilizers suitable for use herein include, but are not limited to polyethylene glycols such as PEG 40 or PEG 600; zinc salts such as zinc citrate; polyoxyalkylene block-polymers (e.g., Pluronics); aminocarboxylic acids or salts thereof; glycerols; dyes such as Blue #1 or Green #3; phosphates such as phosphoric acid, sodium phosphate or sodium acid pyrophosphate; stannous salts such as stannous chloride; sodium stannate; citric acid; etidronic acid; carbomers or carboxypolymethylenes such as those of the Carbopol® seriers, butylated hydroxytoluene (BHT), ethylenediaminetetraacetic acid (EDTA) and mixtures thereof.

Anti-tartar agents useful herein include: phosphates. Phosphates include pyrophosphates, polyphosphates, polyphosphonates and mixtures thereof. Pyrophosphates are among the best known for use in dental care products. Pyrophosphate ions delivered to the teeth derive from pyrophosphate salts. The pyrophosphate salts useful in the present compositions include the dialkali metal pyrophosphate salts, tetra-alkali metal pyrophosphate salts, and mixtures thereof. Disodium dihydrogen pyrophosphate (Na₂H₂P₂O₇), tetrasodium pyrophosphate (Na₄P₂O₇), and tetrapotassium pyrophosphate (K₄P₂O₇) in their unhydrated as well as hydrated forms are preferred. Anticalculus phosphates include potassium and sodium pyrophosphates; sodium tripolyphosphate; diphosphonates, such as ethane-1-hydroxy-1,1-diphosphonate; 1-azacycloheptane-1,1-diphosphonate; and linear alkyl diphosphonates; linear carboxylic acids and sodium and zinc citrate.

Agents that may be used in place of or in combination with the pyrophosphate salt include materials such as synthetic anionic polymers including polyacrylates and copolymers of maleic anhydride or acid and methyl vinyl ether (e.g. Gantrez, as described, for example, in U.S. Pat. No. 4,627,977, to Gaffar et al. herein incorporated by reference in its entirety, as well as e.g. polyamino propane sulfonic acid (AMPS), zinc citrate trihydrate, polyphosphates (e.g. tripolyphosphate; hexametaphosphate), diphosphonates (e.g. EHDP, AMP), polypeptides (such as polyaspartic and polyglutamic acids), and mixtures thereof.

One of more fluoride ion sources incorporated into the film compositions as anticaries agents. Fluoride ions are included in many oral care compositions for this purpose, and similarly may be incorporated in the invention in the same way. Detailed examples of such fluoride ion sources can be found in U.S. Pat. No. 6,121,315 to Nair et al., herein incorporated by reference in its entirety.

Also useful herein are tooth desensitizing agents. Tooth desensitizing agents that may be used in the present invention include potassium nitrate, citric acid, citric acid salts, strontium chloride, and the like, as well as other desensitizing agents known in the art. The amount of desensitizing agent included within the dental whitening compositions of the present invention may vary according to the concentration of the potassium nitrates, the desired strength and intended treatment times. Accordingly, if included at all, the other desensitizing agents will preferably be included in an amount in a range from about 0.1% to about 10% by weight of the dental desensitizing composition, more preferably in a range from about 1 to about 7% by weight of the wet film composition.

Antimicrobial agents can also be present in the film compositions of the present invention as oral agents or topical skin and/or systemic actives. Such agents may include, but are not limited to, 5-chloro-2-(2,4-dichlorophenoxy)-phenol, commonly referred to as triclosan, chlorhexidine, alexidine, hexetidine, sanguinarine, benzalkonium chloride, salicylamide, domiphen bromide, cetylpyridium chloride (CPC), tetradecyl pyridinium chloride (TPC); N-tetradecyl-4-ethyl pyridinium chloride (TDEPC); octenidine; delmopinol, octapinol, and other piperidino derivatives, niacin preparations; zinc/stannous ion agents; antibiotics such as AUGMENTIN, amoxyicillin, tetracycline, doxycyline, minocycline, and metronidazole; and analogs, derivatives and salts of the above antimicrobial agents and mixtures thereof.

Anti-inflammatory agents can also be present in the film compositions of the present invention as oral agents or topical skin and/or systemic actives. Such agents may include, but are not limited to, non-steroidal anti-inflammatory agents or NSAIDs, such as propionic acid derivatives; acetic acid derivatives; fenamic acid derivatives; biphenylcarboxylic acid derivatives; and oxicams. All of these NSAIDS are fully described in U.S. Pat. No. 4,985,459 to Sunshine et al., issued Jan. 15, 1991, incorporated by reference herein in its entirety. Examples of useful NSAIDS include acetyl salicylic acid, ibuprofen, naproxen, benoxaprofen, flurbiprofen, fenoprofen, fenbufen, ketoprofen, indoprofen, pirprofen, carprofen, oxaprozin, pranoprofen, microprofen, tioxaprofen, suprofen, alminoprofen, tiaprofenic acid, fluprofen, bucloxic acid and mixtures thereof. Also useful are the steroidal anti-inflammatory drugs such as hydrocortisone and the like, and COX-2 inhibitors such as such as meloxicam, celecoxib, rofecoxib, valdecoxib, etoricoxib or mixtures thereof. Mixtures of any of the above antiinflammatories may be used.

Anesthetic agent may also be incorporated herein. Examples of suitable anesthetic agents include, but are not limited to, benzocaine, betoxycaine, biphenamine, bupivacaine, butacaine, dibucaine hydrochloride, dyclonine, lidocaine, mepivacaine, procaine, propanidid, propanocaine, proparacaine, propipocaine, propofol, propoxycaine hydrochloride, pseudococaine, tetracaine hydrochloride and mixtures thereof.

Upper respiratory actives can also be used herein. Examples of such actives are sympathomimetic agents administered systemically or topically for decongestant use, including propylhexedrine, phenylephrine, phenylpropanolamine, pseudoephedrine, naphazoline hydrochloride, oxymetazoline hydrochloride, tetrahydrozoline hydrochloride, xylometazoline hydrochloride, and ethylnorepinephrine hydrochloride; anti-histamines are chlorpheniramine, brompheniramine, clemastine, ketotifen, azatadine, loratadine, terfenadine, cetirizine, astemizole, tazifylline, levocabastine, diphenhydramine, temelastine, etolotifen, acrivastine, azelastine, ebastine, mequitazine, mizolastine, levocetirizine, mometasone furoate, carebastine, ramatroban, desloratadine, noberastine, selenotifen, alinastine, efletirizine, tritoqualine, norastemizole, tagorizine, epinastine, acrivastine and mixtures thereof; antitussives such as dextromethorphan, benzonatate, and guifenecin and mixtures thereof. Other useful upper respiratory actives and be found in U.S. Pat. No. 4,619,934, herein incorporated by reference in its entirety.

Gastro-intestinal actives can also be incorporated. Examples of suitable gastro-intestinal actives include anticholinergics, including: atropine, clidinium and dicyclomine; antacids, including aluminum hydroxide, basic bismuth salts such as bismuth subsalicylate, bismuth ranitidine citrate, bismuth subcitrate, bismuth subnitrate, aluminum or bismuth salts of polysulfated saccharides such as aluminum sucrose octasulfate or bismuth sucrose octasulfate, simethicone, calcium carbonate and magaldrate (other examples of antacids can be found in 21CFR 331.11 which is incorporated herein by reference); H (2)-receptor antagonists, including cimetidine, famotidine, nizatidine and ranitidine; laxatives, including: bisacodyl, picosulfate, and casanthrol (other examples of laxatives can be found in the Federal Registry, Vol. 50, No. 10, Jan. 15, 1985, pp. 2152-58, which is incorporated herein by reference); gastroprotectants, including sucralfate and sucralfate humid gel; gastrokinetic and prokinetic agents including cisapride, metoclopramide and eisaprode; proton pump inhibitors including omeprazole, lanzoprazole, and antidiarrheals including: diphenoxylate and loperamide; agents which are bacteriostatic or bactericidal to the ulcer-inducing organism Heliobacter pylori such as amoxicillin, metronidazole, erythromycin, or nitrofurantoin and others agents for treating H. pylori disclosed in U.S. Pat. No. 5,256,684, which is incorporated herein by reference in its entirety; polyanionic materials useful for the treatment of ulcers and other gastrointestinal disorders including amylopectin, carragemum, sulfated dextrins, inositol hexaphosphate, or other similar agents and mixtures thereof.

Nutrients may improve the condition of the oral cavity and can be included in the oral care substances or compositions of the present invention. Examples of nutrients include minerals, vitamins, oral nutritional supplements, enteral nutritional supplements, and mixtures thereof.

Smoking cessation agents such as nicotine may also be incorporated in the film compositions of the present invention.

An individual enzyme or combination of several compatible enzymes can also be included in the oral care substance or composition of the present invention.

Enzymes are biological catalysts of chemical reactions in living devices. Enzymes combine with the substrates on which they act forming an intermediate enzyme substrate complex. This complex is then converted to a reaction product and a liberated enzyme which continues its specific enzymatic function.

Enzymes provide several benefits when used for cleansing of the oral cavity. Proteases break down salivary proteins which are absorbed onto the tooth surface and form the pellicle; the first layer of resulting plaque. Proteases along with lipases destroy bacteria by lysing proteins and lipids which form the structural component of bacterial cell walls and membranes. Dextranases break down the organic skeletal structure produced by bacteria that forms a matrix for bacterial adhesion. Proteases and amylases, not only present plaque formation, but also prevent the development of calculus by breaking-up the carbohydrate protein complex that binds calcium, preventing mineralisation. Enzymes useful in the present invention include any of the commercially available proteases, glucanohydrolases, endoglycosidases, amylases, mutanases, lipases and mucinases or compatible mixtures thereof. Preferred are the proteases, dextranases, endoglycosidases and mutenases, most preferred being papain, endoglycidase, lysozyme, or a mixture of dextranase and mutanase.

Other materials that can be used with the present invention include commonly known mouth and throat products. These products include, but are not limited to anti-fungal, antibiotic and analgesic agents. Antioxidants are generally recognized as useful in compositions such as those of the present invention. Antioxidants that may be included in the oral care composition or substance of the present invention include, but are not limited to Vitamin E, ascorbic acid, Uric acid, carotenoids, Vitamin A, flavonoids and polyphenols, herbal antioxidants, melatonin, aminoindoles, lipoic acids and mixtures thereof.

Histamine-(H-2)receptor antagonist compounds (H-2 antagonists) may be used in the oral care composition of the present invention. As used herein, selective H-2 antagonists are compounds that block H-2 receptors, but do not have meaningful activity in blocking histamine-(H-1) receptors.

Additional useful actives can be found in U.S. Pat. No. 6,638,528 herein incorporated by reference in its entirety.

An additional carrier material may also be added to the film composition of the present invention. These materials can be added as additional components for properties other than those previously mentioned and can include humectants and include glycerin, sorbitol, polyethylene glycol and the like. The film composition may comprise the active substance itself, together with one or more active substance enhancers, for example catalysts and/or potentiators to modify the release and/or activity of the active substance.

The film compositions of the invention may additionally comprise additional substances such as flavors, colors, etc. which may for example be deposited onto the surface of the film or impregnated into the bulk of the film. The topical or system active is preferably teeth whitening substance. The teeth whitening substance can take the form of a peroxide-containing gel. Suitable gels may be based on glycerol containing a peroxide such as hydrogen peroxide or an organic peroxide. A suitable gel is that disclosed in U.S. Pat. No. 3,657,413, for example that sold under the trade mark PROXIGEL by The Block Drug Company (USA) (since acquired by GlaxoSmithKline plc). Other suitable peroxide-containing gels are for example disclosed in the art references cited above. The film may have the topical or system active deposited upon its surface.

A pH adjusting agent may also be added to optimise the storage stability of the gel and to make the substance safe for the oral tissues. These pH adjusting agents, or buffers, can be any material which is suitable to adjust the pH of the oral care substance. Suitable materials include sodium bicarbonate, sodium phosphate, sodium hydroxide, ammonium hydroxide, sodium stannate, triethanolamine, citric acid, hydrochloric acid, sodium citrate, and combinations thereof. The pH adjusting agents are added in sufficient amounts so as to adjust the pH of the substance or composition to a suitable value, e.g. about 4.5 to about 11, preferably from about 5.5 to about 8.5, and more preferably from about 6 to about 7. The pH adjusting agents are generally present in an amount of from about 0.01% to about 15% and preferably from about 0.05% to about 5%, by weight of the oral care substance.

For example a gel may be deposited directly as a layer on a surface of a film layer as described above. Alternatively a gel may be absorbed into the above-described film layer, or impregnated into the bulk of the film material, or deposited between layers of a multiple layered film.

Methods of depositing substances upon the surfaces of film materials as described above are known, for example printing, e.g. silo screen printing, passing between impregnated rollers, dosing, a pump and nozzle, spraying, dipping etc. Methods of impregnating substances into the bulk of film materials are also known, for example admixing the substance into the strip material and then forming the strip, or exposure of the strip to the substance under conditions which cause the substance to be impregnated into the strip. Alternatively, one example of the film material may be a foam material, particularly an open-cell foam material, and the substance may be impregnated into the strip material by introducing the substance into the cells of the foam.

The device of the invention may be marked with one or more visible symbol, e.g. text matter, a trade mark, a company logo, an area of color, or an alignment feature such as a visible line or notch etc. to assist the user in applying the device to the teeth in a proper alignment. Such an alignment feature may for example comprise a symbol to show the user which way up the device should be whilst applying the device to the teeth, or which of a pair of the devices is intended for the upper teeth and which for the lower teeth. This way the device may be made more visually attractive and/or easier to use. Such symbol(s) may be applied by conventional printing processes, e.g. silk screen printing, inkjet printing etc. to the surface of the plastically deformable material opposite to the surface on which is attached the layer of an absorbent material.

If such a visible symbol is applied to this surface, a cover layer can, optionally, be applied over the symbol, for example to protect it. This cover layer may be transparent or translucent to allow visible symbols to be seen through this layer. Such a cover layer can, optionally, be applied to the film by pressing, e.g. rolling, the material of the cover layer in contact with the film.

Methods for Delivering Topical and Systemic Actives

In certain embodiments, the present invention can be used where retention of the topical or systemic active is required for topical activity or adequate systemic absorption. The film compositions of the present invention are particularly useful for whitening tooth surfaces. Generally, the delivery of the teeth whitening actives involves topically applying the inventive film containing a safe and effective amount of such actives to a tooth or teeth and gums in a manner described in U.S. Pat. Nos. 5,894,017; 5,891,453; 6,045,811; and 6,419,906, each of which is herein incorporated by reference in its entirety. The frequency of application and the period of use will vary widely depending upon the level of treatment required or desired, e.g., the degree of teeth whitening and/or degree of topical wound healing/disinfection desired.

When applied as a patch for skin or mucosa, the films of the present invention can be useful for problem skin areas needing more intensive treatment or for the transdermal delivery of drugs. The patch can be occlusive, semi-occlusive or non-occlusive. The topical or systemic actives of the present invention can be contained within or coated on the surface of the film or be applied to the skin prior to application of the film. The film can also include actives such as chemical initiators for exothermic reactions such as those described in PCT application WO 9701313 to Burkett et al. Optionally, the film can be applied at night as a form of night therapy. Examples of useful transdermal systems are described in U.S. Pat. Nos. 3,598,122; 3,598,123; 3,731,683; 3,797,494; 4,286,592; 4,314,557; 4,379,454; 4,435,180; 4,559,222; 4,568,343; 4,573,999; 4,588,580; 4,645,502; 4,704,282; 4,816,258; 4,849,226; 4,908,027; 4,943,435; and 5,004,610, all of which are herein incorporated by reference in their entirety. Actives commonly associated with transdermal delivery are disclosed in U.S. Pat. Nos. 5,843,468 and 5,853,751, both of which are herein incorporated by reference in their entirety.

### EXAMPLES

The film compositions illustrated in following examples illustrate specific embodiments of the film compositions of the present invention, but are not intended to be limiting thereof. Other modifications can be undertaken by the skilled artisan without departing from the spirit and scope of this invention.

All exemplified film compositions can be prepared by conventional formulation and mixing techniques. Component amounts are listed as weight percents and exclude minor materials such as diluents, filler, and so forth. The listed formulations, therefore, comprise the listed components and any minor materials associated with such components.

### Example I

The following is an example of a multi-layer film of the present invention.

| Ingredient | Amount (weight percent) | |
|---|---|---|
| Layer 1 | | |
| Hydroxypropyl cellulose¹ | 2% | w/w |
| Calcium Chloride anhydrous | 0.5% | w/w |
| Purified Water, USP/EP | 97.5% | w/w |
| Layer 2 | | |
| Povidone, USP K-90² | 15% | w/w |
| Sodium Alginate³ | 2% | w/w |
| Purified water, USP/EP | 83% | w/w |

¹Hydroxypropyl cellulose, Klucel M CS, Hercules Inc., Wilmington DE.
²Polyvinylpyrrolidone USP K-90, International Specialties Products(ISP), Wayne, NJ.
³Sodium Alginate, Pronova UP MVG, FMC Biopolymers, Philadelphia, PA.

In a suitable container (A) water, calcium chloride and hydroxyethyl cellulosde are mixed until a homogeneous solution is formed.

In a separate container (B) water, povidone, and alginate are mixed until dissolved and uniform.

The contents of container A is then cast at desired thickness on a non-stick surface at room temperature to form the first layer of the inventive multi-layer film. The cast layer can optionally be dried under warm air flow.

The contents of container B is then cast at desired thickness over the above described first layer at room temperature to form the second layer of the multi-layer film. The cast layer can optionally be dried under warm air flow.

### Example II

The following is an example of multii-layer anesthetic film of the present invention.

| Ingredient | Amount (weight percent) | |
|---|---|---|
| Layer 1 | | |
| Hydroxypropyl methylcellulose¹ | 1% | w/w |
| Chitosan² | 2% | w/w |
| Purified Water, USP/EP | 97% | w/w |
| Layer 2 | | |
| PVP/VA Copolymer³ | 20% | w/w |
| Carboxymethyl cellulose⁴ | 2% | w/w |
| Purified water, USP/EP | 53% | w/w |
| Anesthetic agent base (lidocaine base) | 25% | w/w |

¹Hydroxypropyl methylcellulose, Benecel MP874, Hercules Inc., Wilmington DE.
²Chitosan, Protasan UP CL 113, FMC Biopolymers, Philadelphia, PA.
³PVP/VA Copolymer, Plasdone S-630, International Specialties Products(ISP), Wayne, NJ.
⁴Carboxymethyl cellulose, Aqualon CMC 9M, Hercules Inc., Wilmington DE.
⁵ALB CG 35% hydrogen peroxide solution, Atofina, Philadelphia, Pa.

In a suitable container (A) water, chitosan, and hydroxypropyl methylcellulosde are mixed until a homogeneous solution is formed.

In a separate container (B) water, Plasdone, carboxymethyl cellulose, and lidocaine base are mixed until dissolved and uniform.

The contents of container A is then cast at desired thickness on a non-stick surface at room temperature to form the first layer of the inventive multi-layer, anesthetic film. The cast layer can optionally be dried under warm air flow.

The contents of container B is then cast at desired thickness over the above described first layer at room temperature to form the second layer of the multi-layer, anesthetic film. The cast layer can optionally be dried under warm air flow.

### Example III

The following is an example of a muli-layer, teeth whitening film of the present invention.

| Ingredient | Amount (weight percent) | |
|---|---|---|
| Layer 1 | | |
| Hydroxypropyl methylcellulose¹ | 1.2% | w/w |
| Sodium Lauryl Sulfate | 0.3% | w/w |
| Purified Water, USP/EP | 98.5% | w/w |
| Layer 2 | | |
| Povidone, USP K-90² | 16% | w/w |
| Sodium Phosphate Dibasic | 0.2% | w/w |
| Purified water, USP/EP | 76% | w/w |
| Hydrogen Peroxide 35%³ | 7% | w/w |
| Glycerin, USP | 0.8% | w/w |

¹Hydroxypropyl methylcellulose, Benecel MP874, Hercules Inc., Wilmington DE.
²Polyvinylpyrrolidone, USP K-90, International Specialties Products(ISP), Wayne, NJ.
³ALB CG 35% hydrogen peroxide solution, Atofina, Philadelphia, Pa.

In a suitable container (A) water, sodium lauryl sulfate, and hydroxypropyl methylcellulosde are mixed until a homogeneous solution is formed.

In a separate container (B) water, Povidone, sodium phosphate dibasic, glycerin, and hydrogen peroxide are mixed until dissolved and uniform.

The contents of container A is then cast at desired thickness on a non-stick surface at room temperature to form the first layer of the inventive multi-layer, teeth whitening film. The cast layer can optionally be dried under warm air flow.

The contents of container B is then cast at desired thickness over the above described first layer at room temperature to form the second layer of the multi-layer, teeth whitening film. The cast layer can optionally be dried under warm air flow.

### Example IV

The following is an example of a multi-layer, teeth whitening film of the present invention.

| Ingredient | Amount (weight percent) | |
|---|---|---|
| Layer 1 | | |
| Povidone, USP K-90¹ | 14% | w/w |
| Sodium Lauryl Sulfate | 0.6% | w/w |
| Hydrogen Peroxide 35%² | 8% | w/w |
| Purified Water, USP/EP | 77.4% | w/w |
| Layer 2 | | |
| Chitosan³ | 2% | w/w |
| Purified water, USP/EP | 98% | w/w |

¹Polyvinylpyrrolidone, USP K-90, International Specialties Products(ISP), Wayne, NJ.
²ALB CG 35% hydrogen peroxide solution, Atofina, Philadelphia, Pa.
³Chitosan, Protasan UP CL 113, FMC Biopolymers, Philadelphia, PA.

In a suitable container (A) water, Sodium Lauryl Sulfate, Hydrogen Peroxide, and Povidone are mixed until a homogeneous solution is formed.

In a separate container (B) water and Chitosan are mixed until dissolved and uniform.

The contents of container A is then cast at desired thickness on a non-stick surface at room temperature to form the first layer of the inventive multi-layer, teeth whitening film. The cast layer can optionally be dried under warm air flow.

The contents of container B is then cast at desired thickness over the above described first layer at room temperature to form the second layer of the multi-layer, teeth whitening film. The cast layer can optionally be dried under warm air flow.

### Example V

The following is an example of a multi-layer, teeth whitening film of the present invention.

| Ingredient | Amount (weight percent) | |
|---|---|---|
| Layer 1 | | |
| PVP/VA Copolymer¹ | 10% | w/w |
| Poloxamer² | 5% | w/w |
| Cetyl Pyridinium Chloride³ | 0.2% | w/w |
| Hydrogen Peroxide 35%⁴ | 6% | w/w |
| Purified Water, USP/EP | 78.8% | w/w |
| Layer 2 | | |
| Carboxymethyl cellulose⁵ | 1% | w/w |
| Purified water, USP/EP | 99% | w/w |

¹PVP/VA Copolymer, Plasdone S-630, International Specialties Products(ISP), Wayne, NJ.
²Poloxamer, Pluracare L-44 NF, BASF Corporation, Mount Olive, NJ.
³Cetyl Pyridinium Chloride, Spectrum Laboratory Products, Gardena, CA.
⁴ALB CG 35% hydrogen peroxide solution, Atofina, Philadelphia, Pa.
⁵Carboxymethyl cellulose, Aqualon CMC 9H, Hercules Inc., Wilmington DE.

In a suitable container (A) water, Cetyl Pyridinium Chloride, Pluracare, Hydrogen Peroxide, and Plasdone are mixed until a homogeneous solution is formed.

In a separate container (B) water and Carboxymethyl Cellulose are mixed until dissolved and uniform.

The contents of container A is then cast at desired thickness on a non-stick surface at room temperature to form the first layer of the inventive multi-layer film. The cast layer can optionally be dried under warm air flow.

The contents of container B is then cast at desired thickness over the above described first layer at room temperature to form the second layer of the multi-layer, teeth whitening film. The cast layer can optionally be dried under warm air flow.

1. A multi-layer, stand alone, film composition, comprising:
a.) a first water-soluble layer comprising:
i.) a neutral water soluble polymer; and
ii.) a polyvalent cationic ion source; and
b.) a second water-soluble layer comprising an anionic water soluble polymer wherein the multi-layer film composition dissolves or disperses in water at a rate slower than any of the individual water-soluble layers.

2. A film composition according to claim 1, wherein the neutral water-soluble polymer is selected from the group consisting of hydroxybutyl cellulose, hydroxylethylmethyl cellulose, hydroxyethyl cellulose, hydroxypropyl cellulose, polyvinylpyrrolidone, polyvinylpyrrolidone/vinyl acetate copolymer, hydroxypropyl methylcellulose, methylcellulose, polyethyleneglycol, polyvinyl alcohol and mixtures thereof.

3. A film composition according to claim 1, wherein the polyvalent cationic ion source is selected from the group consisting of aluminum salts, chromium salts, calcium salts, zinc salts, magnesium salts, iron salts, barium salts, manganese salts, stannous salts and mixtures thereof.

4. A film composition according to claim 1, wherein the anionic water soluble polymer is selected from the group consisting of sodium alginate, pectin, carrageenan, carboxymethyl cellulose, sodium polyacrylate, sodium polymetacrylate, sodium maleate/methyl vinyl ether copolymer and mixtures thereof.

5. A multi-layer, stand alone, film composition, comprising:
a.) a first water-soluble layer comprising a cationic polymer; and
b.) a second water-soluble layer comprising an anionic polymer wherein the multi-layer film composition dissolves or disperses in water at a rate slower than any of the individual water-soluble layers.

6. A film composition according to claim 5, wherein the cationic water soluble polymer is selected from the group consisting of chitosan, polyquatemium-10, polyquaternium-11, polyquatemium-16, polyquaternium 24, polyquaternium-28, polyquaternium-44, polyquaternium-46, polyvinylpyrrolidone/dimethylaminopropyl methacrylamide copolymer and mixtures thereof.

7. A film composition according to claim 5, wherein the anionic water soluble polymer is selected from the group consisting of sodium alginate, pectin, carrageenan, carboxymethyl cellulose, sodium polyacrylate, sodium polymethacrylate, sodium maleate/methyl vinyl ether copolymer and mixtures thereof.

8. A multi-layer, stand alone, film composition, comprising:
a.) a first water-soluble layer comprising:
i.) a neutral water soluble polymer; and
ii.)an anionic or cationic surfactant; and
b.) a second water-soluble layer comprising:
i.) a neutral water soluble polymer;
ii.) water-soluble salt wherein the multi-layer film composition dissolves or disperses in water at a rate slower than any of the individual water-soluble layers.

9. A film composition according to claim 8, wherein the neutral water-soluble polymer of the first water-soluble layer is selected from the group consisting of hydroxyethyl cellulose, hydroxypropyl cellulose, polyvinylpyrrolidone, polyvinylpyrrolidone/vinyl acetate copolymer, hydroxypropyl methylcellulose, methylcellulose, polyethyleneglycol, polyethylene oxide, polyethylene oxide/polypropylene oxide block copolymer, polyvinyl alcohol and mixtures thereof.

10. A film composition according to claim 8, wherein the anionic surfactant is selected from the group consisting of sodium lauryl sulfate, sodium dodecyl benzenesulfonate, sodium C12-15 pareth-15 sulfonate, sodium methyl cocoyl taurate, disodium lauryl sulfosuccinate and mixtures thereof.

11. A film composition according to claim 8, wherein the cationic surfactant is selected from the group consisting of cetyl pyridinium chloride, benzalkonium chloride, dodecyl trimethylammonium chloride and mixtures thereof.

12. A film composition according to claim 8, wherein the water-soluble salt is selected from the group consisting of sodium chloride, potassium chloride, magnesium chloride, calcium chloride, zinc chloride, sodium acetate, sodium fluoride, sodium phosphate, potassium phosphate, sodium citrate, sodium oxalate, acidic salts thereof and mixtures thereof.

13. A multi-layer, stand alone, film composition, comprising:
a.)a first water-soluble layer comprising:
i.) a neutral water soluble polymer; and
ii.) an anionic surfactant; and
b.) a second water-soluble layer comprising a cationic water soluble polymer wherein the multi-layer film composition dissolves or disperses in water at a rate slower than any of the individual water-soluble layers.

14. A film composition according to claim 13, wherein the neutral water-soluble polymer is selected from the group consisting of hydroxyethyl cellulose, hydroxypropyl cellulose, polyvinylpyrrolidone, polyvinylpyrrolidone/vinyl acetate copolymer, hydroxypropyl methylcellulose, methylcellulose, polyethyleneglycol, polyethylene oxide, polyethylene oxide/polypropylene oxide block copolymer, polyvinyl alcohol and mixtures thereof.

15. A film composition according to claim 13, wherein the anionic surfactant is selected from the group consisting of sodium lauryl sulfate, sodium dodecyl benzenesulfonate, sodium C12-15 pareth-15 sulfonate, sodium methyl cocoyl taurate, disodium lauryl sulfosuccinate and mixtures thereof.

16. A film composition according to claim 13, wherein the cationic water soluble polymer is selected from the group consisting of chitosan, polyquatemium-10, polyquatemium-11, polyquatemium-16, polyquatemium 24, polyquatemium-28, polyquaternium-44, polyquatemium-46, polyvinylpyrrolidone/dimethylaminopropyl methacrylamide copolymer and mixtures thereof.

17. A film composition, comprising:
a first water-soluble layer a cationic surfactant; and
b.) a second water-soluble layer comprising an anionic water soluble polymer;
wherein the multi-layer film composition dissolves or disperses in water at a rate slower than any of the mixtures thereof.

18. A film composition according to claim 17, wherein the neutral water-soluble polymer is selected from the group consisting of hydroxyethyl cellulose, hydroxypropyl cellulose, polyvinylpyrrolidone, polyvinylpyrrolidone/vinyl acetate copolymer, hydroxypropyl methylcellulose, methylcellulose, polyethyleneglycol, polyethylene oxide, polyethylene oxide/polypropylene oxide block copolymer, polyvinyl alcohol and mixtures thereof.

19. A film composition according to of claim 17, wherein the cationic surfactant is selected from the group consisting of sodium chloride, potassium chloride, dodecyl trimethylammonium chloride and mixtures thereof.

20. A film composition according to claim 17, wherein the anionic water soluble polymer is selected from the group consisting of sodium alginate, pectin, carrageenan, carboxymethyl cellulose, sodium polyacrylate, sodium polymethacrylate, sodium maleate/methyl vinyl ether copolymer and

21. A tri-layer, stand alone, film, comprising:
a.) a first water soluble layer comprising a charged entity;
b.) a second water soluble layer comprising a charged entity; and
c.) a complex layer formed by the interaction of the charged entities of the first and second layers
wherein the tri-layer film composition dissolves or disperses in water at a rate slower than either the first or second water-soluble layers.

22. A method of manufacturing multilayer film compositions, comprising the steps of:
a.) preparing at least one aqueous solution containing a charged first polymer or nonionic polymer comprising a charged species;
b.) casting, spreading or spraying the aqueous solution to form a first polymer layer;
c.) drying the first polymer layer;
d.) preparing a second aqueous solution comprising a second polymer having a charge opposite that of the first polymer or opposite the charged species of the first polymer or the second polymer having a the charged species having a charge opposite that of the first polymer or opposite the charged species the first polymer;
e.) casting, spreading or spraying the second aqueous solution to form a second layer; and
f.) drying the second polymer layer on the first polymer layer to form a multi-layered film.

23. A method of treating the skin, teeth or oral mucosa comprising the step of applying to the skin, teeth or oral mucosa the multi-layered film composition of claim 1.
